(19) United States
(12) Patent Application Publication
(10) Pub. No.: US 2025/0032010 A1
(43) Pub. Date: Jan. 30, 2025

(54) WEARABLE PLASMONIC PAPERFLUIDICS FOR CONTINUOUS BIOFLUID ANALYSIS

(71) Applicant: The Texas A&M University System, College Station, TX (US)

(72) Inventors: Limei Tian, College Station, TX (US); Umesha Mogera, Philadelphia, PA (US); Heng Guo, College Station, TX (US)

(73) Assignee: The Texas A&M University System, College Station, TX (US)

(21) Appl. No.: 18/716,809

(22) PCT Filed: Dec. 6, 2022
(86) PCT No.: PCT/US2022/052056

§ 371 Date: Jun. 5, 2024

Related U.S. Application Data
(60) Provisional application No. 63/286,798, filed on Dec. 7, 2021.

(51) Int. Cl. A61B 5/1455; A61B 5/00; A61B 5/145

(52) U.S. Cl. CPC A61B 5/1455; A61B 5/14517; A61B 5/4266; A61B 5/6833; A61B 2560/0412; A61B 2560/0462

(57) ABSTRACT

In an embodiment, the present disclosure pertains to a wearable sensor. In some embodiments, the wearable sensor includes a double-sided adhesive layer, a paper microfluidic layer, and an encapsulation layer. In an additional embodiment, the present disclosure pertains to a method of biochemical analysis. In general, the method includes collecting biofluid from a subject via a wearable sensor and quantifying the biofluid. In some embodiments, the wearable sensor includes a double-sided adhesive layer and a paper microfluidic layer having a microfluidic channel in a serpentine configuration. In some embodiments the microfluidic channel includes an inlet to receive the biofluid, an outlet to collect the excess biofluid, and a plurality of plasmonic sensors.

### CROSS-REFERENCE TO RELATED APPLICATIONS

This patent application claims priority from, and incorporates by reference the entire disclosure of, U.S. Provisional Patent Application 63/286,798 filed on Dec. 7, 2021.

### STATEMENT REGARDING FEDERALLY SPONSORED RESEARCH

This invention was made with government support under 1648451 awarded by the National Science Foundation and 1R21EB029064-01A1 awarded by the National Institutes of Health. The government has certain rights in the invention.

### TECHNICAL FIELD

The present disclosure relates generally to paperfluidics and more particularly, but not by way of limitation, to wearable plasmonic paperfluidics for continuous biofluid analysis.

### BACKGROUND

This section provides background information to facilitate a better understanding of the various aspects of the disclosure. It should be understood that the statements in this section of this document are to be read in this light, and not as admissions of prior art.

Wearable sweat sensors have the potential to provide clinically important information associated with the health and disease states of individuals. Current sensors mainly rely on enzymes and antibodies as biorecognition elements to achieve specific quantification of metabolite and stress biomarkers in sweat. However, enzymes and antibodies are prone to degrade over time, compromising the sensor performance.

### SUMMARY OF THE INVENTION

This summary is provided to introduce a selection of concepts that are further described below in the Detailed Description. This summary is not intended to identify key or essential features of the claimed subject matter, nor is it to be used as an aid in limiting the scope of the claimed subject matter.

Wearable plasmonic paper-based microfluidic systems for continuous and simultaneous quantitative analysis of sweat loss, sweat rate, and metabolites in sweat are disclosed. Plasmonic sensors based on label-free surface-enhanced Raman spectroscopy (SERS) can provide chemical “fingerprint” information for analyte identification. The wearable systems provide sensitive detection and quantification of uric acid in sweat at physiological and pathological concentrations. The well-defined flow characteristics of paper microfluidic devices enable accurate quantification of sweat loss and sweat rate. The wearable plasmonic devices are soft, flexible, and stretchable, and provide a robust interface with the skin without inducing chemical or physical irritation.

In an embodiment, the present disclosure pertains to a wearable sensor. In some embodiments, the wearable sensor includes a double-sided adhesive layer, a paper microfluidic layer, and an encapsulation layer.

In some embodiments, the paper microfluidic layer includes a microfluidic channel having an inlet, an outlet, and a plurality of plasmonic sensors. In some embodiments plasmonic nanostructures may be selected from the group consisting of metal nanostructures, including but not limited to gold, silver and copper nanospheres, nanorods, nanostars, nanocubes, nanopyramids, and nanowires. In some embodiments, the plasmonic sensors comprise surface ligands selected from the group consisting of antibodies, aptamers, small molecules, Raman reporters, peptides, and organic polymers. In some embodiments, the inlet is configured to receive a fluid that can include, without limitation, biofluid, interstitial fluid, blood, plasma, saliva, urine, sweat, and combinations thereof. In some embodiments, the inlet is a plurality of inlets. In some embodiments, the outlet is configured to connect to an absorbent pad to collect an excess fluid that can include, without limitation, biofluid, interstitial fluid, blood, plasma, saliva, urine, sweat, and combinations thereof. In some embodiments, the microfluidic channel has a serpentine configuration. In some embodiments, the plurality of plasmonic sensors include chromatography paper having a surface-enhanced Raman spectroscopy (SERS) substrate for stable SERS enhancement. In some embodiments, the SERS substrate includes gold nanorods (AuNR). In some embodiments, the encapsulation layer is an optically transparent material. In some embodiments, the encapsulation layer includes at least one of polydimethylsiloxane (PDMS), a silicone adhesive, or a silicone adhesive layer sandwiched between PDMS encapsulation and the paperfluidic layer. In some embodiments, the wearable sensor further includes a laser blocking layer. In some embodiments, the wearable sensor is configured for continuous quantitative analysis of sweat loss, sweat rate, and sweat composition, including pH, ions, amino acids, metabolites, drugs, proteins, and pathogens.

In an additional embodiment, the present disclosure pertains to a method of biochemical analysis. In general, the method includes collecting biofluid from a subject via a wearable sensor and quantifying the biofluid. In some embodiments, the wearable sensor includes a double-sided adhesive layer and a paper microfluidic layer having a microfluidic channel in a serpentine configuration. In some embodiments the microfluidic channel includes an inlet to receive the biofluid, an outlet to collect the excess biofluid, and a plurality of plasmonic sensors.

In some embodiments, the quantifying includes simultaneous quantification of rate and volume of release and concentration of analytes in the biofluid, including pH, ions, metabolites, proteins, and pathogens. In some embodiments, the quantifying includes determining at least one of pH of the biofluid, rate of release from the subject of the biofluid, biofluid loss from the subject, volume of the biofluid, analytes in the biofluid, concentration of analytes in the biofluid, metabolites in the biofluid, concentration of metabolites in the biofluid, or combinations thereof. In some embodiments, the quantifying includes detection of at least one of analytes or metabolites in the biofluid. In some embodiments, the biofluid includes sweat. In some embodiments, the quantifying includes continuous quantitative analysis of sweat loss, sweat rate, and metabolites in sweat. In some embodiments, the quantifying is performed via surface-enhanced Raman spectroscopy (SERS) or other optical approaches, colorimetric assay, enzyme-linked immunosorbent assay, fluorescence-linked immunosorbent assay, and combinations thereof. In some embodiments, the plurality of plasmonic sensors include chromatography paper having a SERS substrate for stable SERS enhancement. In some embodiments, the SERS substrate include gold nanorods (AuNR). In some embodiments, the encapsulation layer includes an optically transparent material. In some embodiments, the encapsulation layer includes at least one of polydimethylsiloxane (PDMS), a silicone adhesive, or a silicone adhesive layer sandwiched between PDMS encapsulation and the paperfluidic layer.

### BRIEF DESCRIPTION OF THE DRAWINGS

A more complete understanding of the subject matter of the present disclosure may be obtained by reference to the following Detailed Description when taken in conjunction with the accompanying Drawings wherein:

FIGS. 1A-1D illustrate wearable plasmonic paperfluidic devices for continuous sweat analysis. FIG. 1A illustrates a wearable plasmonic paperfluidic device positioned on a wrist of a user for sweat collection, storage, and in situ analysis using surface-enhanced Raman spectroscopy (SERS). FIG. 1B is a top view of the paperfluidic device. FIGS. 1C and 1D are exploded assembly views of paperfluidic devices that highlight functional layers.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

Wearable sweat sensors have the potential to provide clinically important information associated with the health and disease states of individuals. Current sensors mainly rely on enzymes and antibodies as biorecognition elements to achieve specific quantification of metabolite and stress biomarkers in sweat. However, enzymes and antibodies are prone to degrade over time, compromising the sensor performance. Disclosed herein is the introduction of a wearable plasmonic paperfluidic system for continuous quantitative analysis of sweat loss, sweat rate, and metabolites in sweat. Plasmonic sensors based on label-free surface-enhanced Raman spectroscopy (SERS) can provide chemical “fingerprint” information for analyte identification. The sensitive detection and quantification of uric acid in sweat at physiological and pathological concentrations is demonstrated. The well-defined flow characteristics of paper microfluidic devices enable accurate quantification of sweat loss and sweat rate. In addition to continuous analysis of sweat, plasmonic paperfluidic devices can also capture and store sweat samples for batch analysis. The wearable plasmonic devices of the present disclosure are soft, flexible, and stretchable, which can robustly interface with the skin without inducing chemical or physical irritation. The plasmonic paperfluidic platform demonstrated herein can be easily adapted for continuous monitoring of various biochemicals in sweat and other peripheral fluids.

Soft, ultrathin skin-interfaced physiological sensors for continuous measurement of physical and chemical biomarkers have broad applications including disease diagnosis, health monitoring, and personalized medicine. Recently developed wearable sweat sensors are capable of analyzing various chemicals in sweat, including electrolytes, metabolites, heavy metals, drugs, and hormones, which can reflect physiological and pathological conditions in the human body. For example, sweat chloride concentration is a standard diagnostic screening tool for cystic fibrosis, and the quantification of sweat glucose has been extensively explored for diabetes management. Similarly, uric acid (UA) is a risk marker for various diseases, including cardiovascular diseases, kidney diseases, and type 2 diabetes. It has been shown that sweat UA concentration is highly correlated with serum concentrations in healthy subjects and patients with gout. To achieve accurate quantification of these biomarkers, wearable sweat sensors require high sensitivity, specificity, and mechanical and environmental stability. Existing sensing modalities mainly rely on electrochemical and colorimetric approaches. These sensors mainly rely on enzymes and antibodies as biorecognition elements to achieve specific quantification of metabolite and stress biomarkers in sweat. For example, enzymes were used for the specific detection of glucose, lactate, uric acid, urea, and ascorbic acid. Antibodies were used for the specific detection of cortisol, a stress biomarker. However, enzymes and antibodies are prone to degrade over time and lose their functionality after exposure to harsh environments and contamination. Therefore, continuous measurements of chemical analytes with high sensitivity, selectivity, and environmental stability remain challenging.

SERS is a highly sensitive analytical method for label-free detection and quantification of a wide range of analytes, including metabolites, macromolecules, and microorganisms. Raman bands of analytes originate from vibrational and rotational modes specific to the molecular structures, which provide chemical “fingerprint” information for analyte identification. However, Raman scattering is very weak as only one in 10⁶-10¹⁰ photons approximately are scattered inelastically. Plasmonic nanostructures can greatly enhance the Raman scattering of analytes near the nanostructure surface by factors of 10⁸ or higher, which enables single-molecule detection. To date, significant progress has been made in the fabrication of various SERS substrates to provide high sensitivity, uniformity, and stability for reliable quantification of analytes at a very low concentration. To further improve sample collection efficiency, flexible SERS substrates, such as plasmonic paper and foams, have been developed. Although SERS offers highly sensitive and specific trace detection, integrating plasmonic sensors with a microfluidic platform for continuous sweat analysis has not been explored. Simultaneous quantification of sweat rate and biomarker concentration is important because these two parameters are intrinsically linked in sweat secretion and reabsorption processes. For instance, the concentration of sodium, chloride, urea, and creatinine changes with changing sweat rate.

Disclosed herein is a wearable plasmonic paperfluidic system that can directly and reliably capture sweat and continuously and simultaneously quantify sweat loss, sweat rate, and the concentration of analytes in sweat in real time. The paper microfluidics enables accurate quantification of sweat loss and sweat rate. The integrated plasmonic nanosensors can detect and quantify UA at physiologically and pathologically relevant concentrations using SERS. The ratiometric SERS approach can reliably quantify UA with variation in the laser power and focus, validated with benchtop and portable Raman spectrometers. Two operation modes of using plasmonic paperfluidic devices to quantify the analytes of varying concentrations, including in situ continuous scans and batch analysis by scanning the samples at the endpoint are demonstrated. The device is soft, thin, flexible, and stretchable, which can interface to the skin without inducing chemical or physical irritation.

### Working Examples

Reference will now be made to more specific embodiments of the present disclosure and data that provides support for such embodiments. However, it should be noted that the disclosure below is for illustrative purposes only and is not intended to limit the scope of the claimed subject matter in any way.

Materials. All chemicals were used as received. Gold (III) chloride trihydrate (>99.9%), silver nitrate (>99%), ascorbic acid (>99.0%), and cetyltrimethylammonium bromide (CTAB, >99%) were obtained from Sigma-Aldrich. Sodium borohydride (98%) and cellulose chromatography paper (Whatman No. 1 grade) were obtained from Fisher-Scientific. Uric acid, glucose, tyrosine, L-phenylalanine (>99%), L-tyrosine (>99%) were obtained from Alfa-Aesar. 10×PBS (ultrapure grade) was obtained from Hoefer. Artificial sweat was obtained from Biochemazone. The purchased artificial sweat does not contain uric acid. Medical-grade double-sided adhesive tape was purchased from 3M. Polydimethylsiloxane (PDMS) elastomer (Sylgard 184) was purchased from Dow Corning. Type 1 deionized (DI) water (18.2 mΩ·cm) was used in all experiments and produced by Sartorius Arium Pro ultrapure water system.

Synthesis of gold nanorods. The AuNRs were prepared using a modified seed-mediated growth method. Briefly, the seed solution was first prepared by mixing 9.75 mL of 0.1 M CTAB and 0.25 mL of 10 mM HAuCl₄ with 0.6 mL of a freshly prepared ice-cold 10 mM NaBH₄ aqueous solution under vigorous stirring at room temperature. Separately, a growth solution was prepared by mixing 95 mL of 0.1 M CTAB with 5 mL of 10 mM HAuCl₄, and then 1 mL of 10 mM silver nitrate solution and 0.55 mL of 0.1 M ascorbic acid. After gently mixing the growth solution, 0.12 mL of the seed solution was added to the growth solution to yield AuNRs. The AuNRs were aged for 12 h to ensure full growth at room temperature. The AuNR solution was centrifuged at 8000 rpm for 10 min to remove excess chemical reagents for further usage.

Preparation of AuNR paper and paperfluidic device. A 4 cm² chromatography-grade filter paper was immersed in the AuNR solution with a longitudinal LSPR intensity of 5 overnight to allow AuNR to be uniformly adsorbed on the paper surface. Subsequently, AuNR paper was washed with DI water to remove loosely bound AuNRs. To remove CTAB from the AuNR surface, AuNR paper was immersed in 10 mM NaBH₄ for 10 minutes, washed with DI water several times, and then dried in a desiccator for 2 hours. Paper fluidic devices with different channel widths of 1 mm to 3 mm were prepared by cutting chromatography paper into desired serpentine shape using a paper cutting tool (Cricut maker) with predesigned AutoCAD patterns. AuNR paper was cut into pieces of 2×2 mm² square or 1 mm diameter circular shape. Finally, double-sided carbon tape, paperfluidic layer, and AuNR paper were assembled on a medical-grade double-sided adhesive tape and then encapsulated with a thin PDMS film. The thickness of PDMS films was varied from 25 to 220 μm to investigate the effect of the PDMS thickness on SERS intensity.

Characterization and measurements. The extinction spectra of AuNR aqueous solution were collected using a UV-vis spectrophotometer (Shimadzu UV-1900). Extinction spectra of AuNR paper were measured using a microspectrophotometer (CRAIC 308PV) connected with a Leica optical microscope (DM4M) with 20× objective in the range of 450-900 nm with 10 accumulations and an ˜0.2 s exposure time in the reflection mode. Transmission electron microscopy (TEM) images were collected with JEOL 1200 EX and AuNR dimensions were estimated from TEM images using ImageJ. Scanning electron microscopy (SEM) images were recorded on an ultrahigh-resolution field emission SEM (JEOL JSM-7500F). Raman spectra were collected with a DXR Raman benchtop spectrometer with a 780 nm wavelength diode laser using laser power of 20 mW and 10× objective. The spectra were measured in the wavelength range of 400-1800 cm⁻¹ with an acquisition time of 60 seconds. Raman spectra were also collected with a portable Wasatch Raman spectrometer with a laser wavelength of 785 nm, laser power of ˜50 mW, and an exposure time of 5 seconds. All experiments involving human subjects were conducted under approval from the Institutional Review Board at Texas A&M University (project number: 118141).

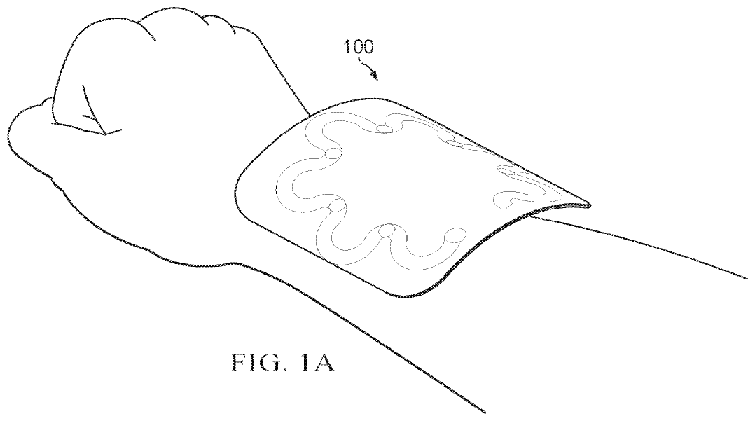

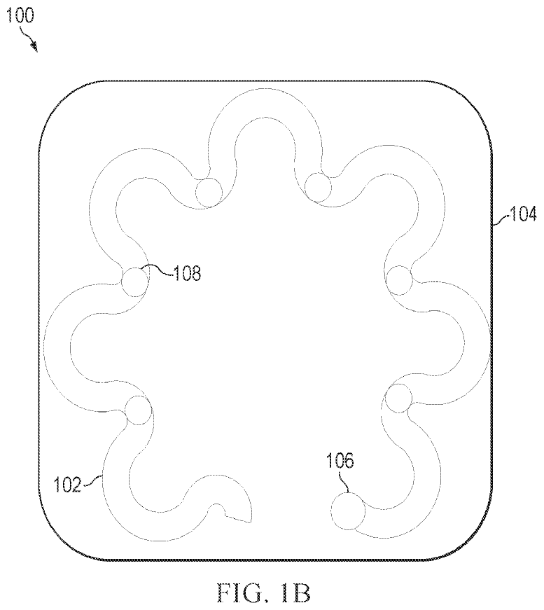

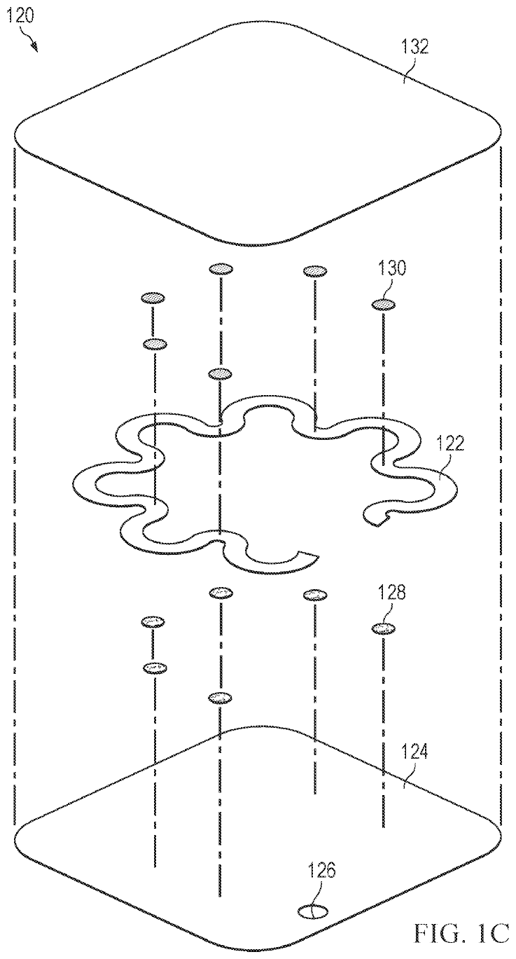

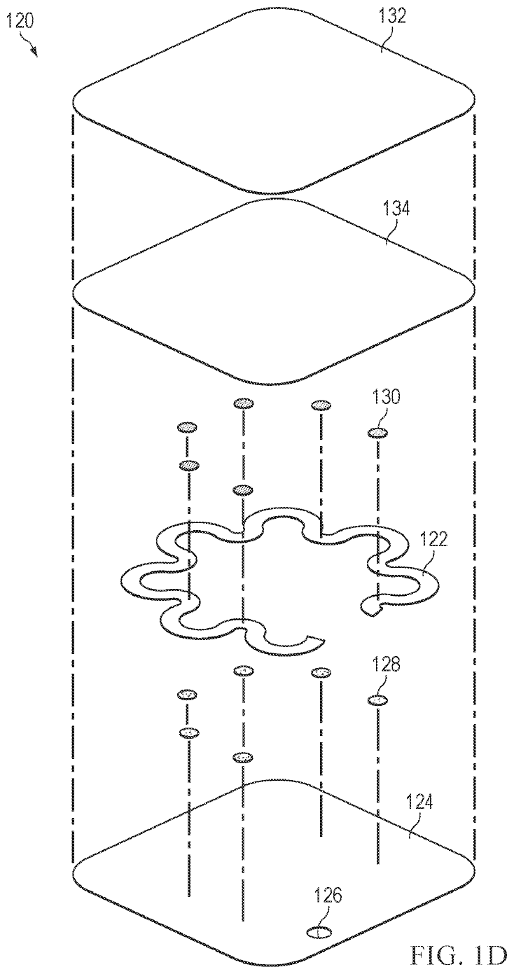

Design of wearable plasmonic paperfluidic device for continuous sweat analysis. FIG. 1A illustrates a soft, ultrathin plasmonic paperfluidic device 100 laminated on a user's wrist for sweat collection, transport, storage, and real-time label-free biochemical analysis with a portable Raman spectrometer. FIG. 1B is a top view of device 100. Device 100 includes a paperfluidic channel 102, an adhesive layer 104, an inlet 106 formed through adhesive layer 104, and a plurality of plasmonic sensors 108. Adhesive layer 104 adheres device 100 to a user's skin and inlet 106 permits sweat from the user's skin to pass therethrough for absorption into paperfluidic channel 102. The absorbed sweat travels along the length of paperfluidic channel 102. FIG. 1C is an exploded assembly of a wearable plasmonic sweat sensor 120 having several functional layers, including a paperfluidic 122, a double-sided adhesive layer 124 that includes an inlet 126, a plurality of laser blockers 128, a plurality of plasmonic sensors 130, and an encapsulation layer 132. FIG. 1D illustrates the wearable plasmonic sweat sensor 120 of FIG. 1C with an additional silicone adhesive layer 134 between encapsulation layer 132 and the layer of plasmonic sensors 130.

The paper microfluidic devices discussed herein have several advantages including (i) cost-effective and easy to dispose of, (ii) simple capture and transportation of biofluids through capillary action without the need of an external pump or force, (iii) absorbency allowing the storage of sensors and samples, (iv) air permeability avoids air bubble problems, (v) high surface area allowing a high-density immobilization of nanoparticles. A cellulose chromatography paper with a serpentine design serves as an effective microfluidic channel that transports the excreted sweat through the porous medium by wicking without the need for external force or inlet pressure. The serpentine design provides paper microfluidic devices flexibility and stretchability and allows the device to accommodate the skin deformation without causing interfacial stress and device degradation. A stretchable, double-sided adhesive forms a mechanically robust interface between the paper microfluidic layer and the skin. An inlet with a diameter of 1 mm opening immediately captures the sweat and avoids the spreading of sweat at the interface and potential contaminations. Plasmonic sensors immobilized at different locations along the paper microfluidic channel quantify the concentration of analytes in the sweat produced at different time points using Raman spectroscopy. Black carbon double-sided adhesive blocks laser and avoids skin damage during Raman spectroscopy measurements. The top PDMS encapsulation layer is optically transparent and exhibits well-defined Raman bands, which can serve as a reference for quantifying the analytes of interest in the sweat. In addition, it minimizes the evaporation of sweat and prevents contamination from the environment.

In some configurations, the encapsulation layer can include a silicone adhesive layer sandwiched between PDMS encapsulation and the paperfluidic layer. Studies conducted identified that the additional adhesive layer can minimize the mixing of analytes of different concentrations because the adhesive forms a conformal contact with the adhesive to minimize the free space. However, as a consequence, the flow rate is slower compared to the configuration without adhesive, and as such, it might not capture the fast flow rate in real-time but provide advantageous properties for certain applications.

As a test, a small droplet of 10 μL water was added to an inlet of a wearable plasmonic paperfluidic device (similar to device 120 of FIGS. 1A and 1B), the fluid passed the first plasmonic sensor within a minute. The color contrast between the wetted and dry regions of the paperfluidic is clearly visible with the eye, allowing the quantification of sweat volume and sweat rate with the position of the fluid front. Plasmonic sensors comprise the chromatography paper uniformed adsorbed with gold nanorods (AuNRs), described as AuNR paper herein. Silver and gold nanostructures are commonly used in SERS due to the enhanced electromagnetic field near the nanostructure surface. Silver nanostructures typically provide higher SERS enhancement and are more cost-effective than gold nanostructures, however, silver is not chemically and environmentally stable, resulting in decreased SERS performance over time. In a design of the present disclosure, AuNR paper as a SERS substrate was chosen for stable SERS enhancement. Gold nanostructures are chemically and environmentally stable. AuNRs offer higher SERS enhancement compared to other gold nanostructures such as gold nanospheres.

AuNRs were synthesized using a seed-mediated method. The immobilization of AuNRs onto the paper is facilitated by the combination of weak interactions, including electrostatic interaction and Van der Waals forces. AuNRs exhibit a uniform dimension distribution of 56.9±3.6 nm in length and 14.5±1.5 nm in diameter, respectively. The extinction of AuNR solution shows two plasmonic bands with peak positions at 511.5 and 765.0 nm corresponding to the transverse and longitudinal localized surface plasmon resonance (LSPR) of AuNRs. The LSPR spectrum of AuNR paper shows a 48.3 nm blue shift in the longitudinal band following the decrease in the refractive index of surrounding media from water to air and cellulose. The shape of the extinction spectrum of AuNR paper remains similar to that of the solution, which suggests the uniform distribution of AuNRs on the paper substrate. The uniformity was further confirmed by SEM. SEM imaging of the pristine paper revealed the heterogeneous morphology of cellulose fibers with diameters from 100 nm to 2 μm. The SEM images of AuNR paper showed uniform distribution of AuNR on the heterogenous paper surface, which is critical for achieving uniform Raman signals from SERS substrates.

Flow characteristics of paper microfluidic devices for sweat loss/rate quantification. For sweat volume and sweat rate quantification, the flow characteristics of the microfluidic serpentine paper sandwiched between top encapsulation PDMS and bottom adhesive layers were characterized. It is hypothesized that the fluid uptake volume of the microfluidic paper is linearly proportional to the paper width and liquid travel distance for a given paper thickness. The travel distance of water with varying known volumes along the serpentine paper with a central length of 109 mm, a thickness of 180 μm, and varying widths of 1 mm, 2 mm, and 3 mm were measured. For 15 μL water introduced to the inlet, the travel distance is around 95 mm, 49 mm, and 33 mm for the paper width of 1 mm, 2 mm, 3 mm, respectively. The total liquid uptake volume of the 1 mm, 2 mm, 3 mm wide paper is 17.5 μL, 35.0 μL, and 52.5 μL, respectively, confirming the linear relationship between the paper width and uptake volume. The integration of AuNR paper with a diameter of 1 mm has a negligible effect on the liquid travel distance. It was observed that travel distance varied linearly with the increase in the liquid volume for all the samples. The calculated slope was 0.16 μL/mm, 0.33 L/mm, and 0.47 μL/mm for the paper with channel widths of 1 mm, 2 mm, 3 mm, respectively, which quantifies the liquid update volume of the microfluidic paper. These calculated values quantify the sweat volume by visualizing the position of the fluid front.

To characterize the liquid wicking kinetics of microfluidic paper, video recordings of continuous flow quantify the travel distance and corresponding time were collected. An increased liquid travel distance in the 2 mm wide serpentine paper was observed when increasing the flow time from 0.7 min to 11.1 min. The liquid travel distance and corresponding liquid uptake volume of the microfluidic paper were measured with varying channel widths of 1 mm to 3 mm with time. The travel time across the same length decreased with the increase in the width of the paper channel. In comparison, the travel time across the entire length of 109 mm is ˜16.9 min for 1 mm wide paper, ˜11.7 min for 2 mm wide paper, ˜8.9 min for 3 mm wide paper. The fluid flow in the microfluidic paper can be modeled by the Lucas-Washburn equation,

$$l = \sqrt{\frac{γ d cos θ t}{4 μ}} ,$$

in which l represents the travel distance, γ is the surface tension of the liquid, d is the average pore radius, μ is the viscosity of the liquid, θ is the contact angle between the fluid and the boundary wall and t represents time. The fitted

$$\sqrt{\frac{γ d cos θ}{4 μ}}$$

is 3.45 mm/sec^{1/2} for 1 mm wide paper, 4.05 mm/sec^{1/2} for 2 mm wide paper, and 4.96 mm/sec^{1/2} for 3 mm wide paper. Using the constant

$$\sqrt{\frac{γ d cos θ}{4 μ}}$$

of 1 mm paper as a reference, a correction factor of

$$0.83 \sqrt{\frac{W}{1 mm}}$$

was introduced to derive the constants of the paper with channel wider than 1 mm. Based on these results, the specific dimension of the microfluidic paper can be chosen to quantify the sweat volume and sweat rate depending on the mounting location of the device on the body (12-120 μL·cm²·h⁻¹).

Plasmonic paperfluidic device design and optimization. Next. AuNR paper sandwiched between a microfluidic paper and top encapsulation PDMS was designed and characterized for highly sensitive detection of uric acid using SERS spectroscopy. Raman spectra of 100 μM uric acid in 1× phosphate-buffered saline (PBS) collected on AuNR paper and pristine paper without AuNR was performed. The Raman bands of uric acids measured from AuNR paper were clearly distinguishable due to the enhancement effect of AuNR while the signals are not detectable from the pristine paper. The prominent peaks observed at 642 cm-1, 895 cm-1, and 1137 cm⁻¹ correspond to the skeletal ring deformation, N—H bending, and C—N stretching of uric acid. These Raman bands are consistent with these measured from uric acid powder. The peak at 496 cm⁻¹ is contributed from the C—N bending and in-plane ring deformation of uric acid and the Si—O—Si stretch of PDMS. To maximize the enhancement of uric acid and minimize the interference from AuNR paper, sodium borohydride (NaBH₄) was employed to remove cetyltrimethylammonium bromide (CTAB) on the AuNR surface. CTAB plays a role in the growth and stabilization of AuNR in an aqueous solution. After AuNR immobilization on paper and extensive water rinsing, strong Raman bands of CTAB with peaks at 754 cm-1 and 1442 cm⁻¹ are still present because of strong interaction between Br and Au. The residual CTAB can be rapidly removed by 10 mM NaBH₄ within 10 minutes. The removal of CTAB stabilizer has a negligible effect on the distribution of AuNR on paper, confirmed with extinction spectra of AuNR paper before and after NaBH₄ treatment.

To quantify the uric acid concentration, the SERS intensity ratio of uric acid and PDMS was employed to minimize the variations in the absolute intensity induced by the variations of laser focus. The SERS spectra of uric acid collected on AuNR paper covered with a quartz slide quantifies the intensity of 496 cm⁻¹ peak is 0.4 times as that of 642 cm⁻¹ peak, therefore a ratiometric intensity I₆₄₂/(I₄₉₆−0.4×I₆₄₂) is used to quantify the sensitivity of uric acid detection in biofluids. The removal of CTAB increases the ratiometric SERS intensity of uric acid by 86%. The thickness of the PDMS encapsulation layer also affects the ratiometric SERS intensity of uric acid, which increases by 1.9 times with decreasing the PDMS thickness from 220 μm to 25 μm. The ratiometric SERS intensities of uric acid collected from different regions of the same sample (2 mm×2 mm) and from samples of different batches yield coefficient of variations of 4% and 3%, respectively, confirming the uniformity of SERS signals.

Sensitive and specific UA detection and continuous quantification. Normalized SERS spectra of uric acids with varying concentrations from 20 μM to 100 μM in 1×PBS were collected from AuNR paper. The concentration range covers the physiological and pathological concentrations in sweat for healthy people and people with gout and hyperuricemia. The ratiometric SERS intensity of UA linearly increases with the increase in the concentration. The SERS signals were obtained from the concentration of UA as low as 1 μM. The ratiometric SERS intensity of UA was evaluated with varying pH over a medically relevant range from pH 5.5 to pH 7.4. The ratiometric intensity of uric acid at pH 6.5 and pH 7.4 is slightly higher than that at pH 5.5 and the coefficient of variation is around 10%. To evaluate the specificity of SERS signals for UA detection, AuNR paper was exposed to several potential interfering molecules, including tyrosine, glucose, ascorbic acid, and phenylalanine, at the concentration of 100 μM. The SERS spectra of these interfering molecules confirm the absence of Raman bands at 642 cm⁻¹, therefore their presence in biofluids does not affect the accuracy of UA quantification. The specific detection of UA was further confirmed by exposing AuNR paper to 100 μM UA in artificial sweat, which includes amino acids, minerals, and various metabolites and simulates the composition and properties of the real human eccrine sweat (Biochemazone™). The ratiometric intensity of UA in PBS and artificial sweat shows negligible difference confirming the specificity.

Two operation modes of using plasmonic paperfluidic devices to quantify the analytes of varying concentrations were demonstrated. The first mode involves a continuous scan of one sensor to quantify the rapidly changing concentration of UA. In continuous scan mode, the fluid uptake volume can be extended by interfacing the outlet of the paperfluidic device to a cellulose wicking pad. The UA solutions of 30 μL with changing concentrations of 20 μM and 100 μM were sequentially introduced to the inlet of the paperfluidic device while the SERS spectra were continuously collected from the sensor. The first spectrum at 0 min was collected right after the 20 μM UA solution of wetted the sensor, which showed a weak 642 cm⁻¹ peak. The ratiometric SERS intensity of UA increased with time and reached a plateau within ˜5 minutes. After 10 minutes, the 100 μM UA solution was introduced and the ratiometric SERS intensity increased and reached another plateau within ˜5 minutes. Next, another cycle of 20 μM and 100 μM UA was introduced and the change in the ratiometric intensity is consistent with the change in the UA concentration.

In the second operation mode, the device has multiple sensors and allows for sample storage and batch analysis by scanning the samples at the endpoint. To demonstrate this capability, 10 μL of 20 μM, 100 μM, and 20 μM UA was introduced in sequence to the inlet of a plasmonic paperfluidic device. Plasmonic sensors are spatially distributed along the paperfluidic channel to quantify the concentration of sequentially introduced UA solutions. The change in the ratiometric SERS intensity follows the change in the UA concentration, confirming the capability of quantifying the time-varying UA concentration.

2.5 Stretchability, flexibility, and application of plasmonic paperfluidic devices. The soft, ultrathin, stretchable double-sided adhesive (180 μm thick) and PDMS (80 μm thick) encapsulated serpentine paperfluidics provide stretchability and flexibility of the wearable devices. All functional components described in FIG. 1C were assembled on a double-sided medical adhesive on a flexible temporary paper liner. After removing the flexible paper liner, a freestanding device with a paperfluidic channel width of 2 mm was mounted on a mechanical stretcher to examine the stretchability. For reference, human skin has a linear response to tensile strain up to 15% and a failure stain at >30%. No delamination between all functional layers in the device under 30% stretch was observed. Under 60% stretch, a small tear appears at the edge of the paperfluidic layer. However, the plasmonic sensors are still in the same location due to the mechanical strain isolation provided by the serpentine paperfluidic layer. Twisted and crumbled paperfluidic devices further demonstrate the flexibility of the soft mechanical construction.

For proof of concept, the performance of plasmonic paperfluidic devices for sweat collection and analysis in healthy human subjects was evaluated. The device can be easily applied and comfortably worn at any location of the body due to its soft mechanical construction. The flexible and stretchable design can accommodate the skin deformation without device delamination and constraints in natural body motions. To enable the application of wearable plasmonic paperfluidic devices in point-of-care settings, the accuracy of the UA quantification using a portable Raman spectrometer in comparison with a standard benchtop system was evaluated. A flexible fiber probe of a portable Raman spectrometer was used to collect SERS spectra from a plasmonic paperfluidic device laminated on the forearm of a healthy human subject. The probe in contact with the sensor was encapsulated to avoid laser exposure. To eliminate laser-induced skin damage, a thin layer of carbon tape was sandwiched between plasmonic paper and double-sided adhesive to completely block a laser power of 65 mW. As expected, the SERS intensity varies with the distance between the laser source and the plasmonic sensor. The SERS intensity reaches a maximum at 0 mm when the laser focuses on the AuNR paper surface and the intensity decreases by 40% and 60% when the laser probe moves up and down by 2 mm, respectively. However, the ratiometric SERS intensity remains the same with the offset laser focus. It was further confirmed that the relative SERS intensity remained the same when the SERS spectra were collected with a benchtop and portable spectrometer. The SERS spectrum of sweat was collected after a healthy human subject wore it and excised for 20 minutes. The human subject did not experience any skin irritation or discomfort during the wear and after the device was detached. The sweat volume collected by the device was around 60 μL. The estimated concentration of UA in sweat is around 28 μM, which is consistent with the concentration of UA in the sweat of healthy individuals reported in the literature.

Conclusions. A wearable plasmonic paperfluidic platform for sweat collection, transport, storage, and continuous, real-time, label-free biochemical analysis has been introduced. The design criteria of paper microfluidic devices to enable accurate quantification of sweat loss and sweat rate have been defined. The integrated SERS nanosensors can provide chemical “fingerprint” information of metabolites in sweat. The sensitive detection and quantification of UA in sweat was demonstrated at concentrations as low as 1 μM, which is below physiological and pathological concentrations in human sweat. The utilization of ratiometric SERS intensity as a quantification approach obviates the need for recalibration with different Raman spectrometers, thus facilitating the broad deployment of wearable plasmonic devices. In addition to continuous in situ analysis of chemicals in sweat, the device can capture and store sweat samples for batch analysis. The device principles and sensing platform can be exploited for continuous analysis of other biomarkers in sweat or other biofluids such as saliva, and interstitial fluids, known to have small sample volumes.

Although various embodiments of the present disclosure have been illustrated in the accompanying Drawings and described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.

1. A wearable sensor comprising:
a double-sided adhesive layer;
a paper microfluidic layer, wherein the paper microfluidic layer comprises a microfluidic channel comprising a plurality of plasmonic sensors; and
an encapsulation layer.

2. (canceled)

3. The wearable sensor of claim 1, wherein the microfluidic channel has a serpentine configuration.

4. The wearable sensor of claim 1, wherein the plurality of plasmonic sensors comprise chromatography paper comprising a surface-enhanced Raman spectroscopy (SERS) substrate for stable SERS enhancement.

5. The wearable sensor of claim 4, wherein the SERS substrate comprises plasmonic nanostructures selected from the group consisting of goldmetal nanostructures.

6. The wearable sensor of claim 5, wherein the goldmetal nanostructures comprise one or more of gold, silver and copper nanospheres, nanorods, nanostars, nanocubes, nanopyramids, and nanowires.

7. The wearable sensor of claim 4, wherein the plasmonic sensors comprise surface ligands selected from the group consisting of antibodies, aptamers, small molecules, Raman reporters, peptides, and organic polymers.

8. The wearable sensor of claim 1, wherein the double-sided adhesive layer comprises an inlet formed therethrough and positioned to overlap the paper microfluidic layer, the inlet being configured to receive a fluid selected from the group consisting of biofluid, interstitial fluid, blood, plasma, saliva, urine, sweat, and combinations thereof.

9. The wearable sensor of claim 1, wherein the double-sided adhesive layer comprises an outlet formed therethrough and positioned to overlap the paper microfluidic layer, the outlet being configured to connect to an absorbent pad to collect an excess fluid from the paper microfluidic layer, the excess fluid being selected from the group consisting of biofluid, interstitial fluid, blood, plasma, saliva, urine, sweat, and combinations thereof.

10. The wearable sensor of claim 1, wherein the encapsulation layer comprises an optically transparent material.

11. The wearable sensor of claim 1, wherein the encapsulation layer comprises at least one of polydimethylsiloxane (PDMS), a silicone adhesive, or a silicone adhesive layer sandwiched between PDMS encapsulation and the paperfluidic layer.

12. The wearable sensor of claim 1, further comprising a laser blocking layer.

13. The wearable sensor of claim 1, wherein the wearable sensor is configured for continuous quantitative analysis of sweat loss, sweat rate, and sweat composition, including pH, ions, amino acids, metabolites, drugs, and proteins.

14. A method of biochemical analysis, the method comprising:
collecting biofluid from a subject via a wearable sensor, wherein the wearable sensor comprises:
a double-sided adhesive layer;
a paper microfluidic layer comprising a microfluidic channel in a serpentine configuration, wherein the microfluidic channel comprises an inlet to receive the biofluid, an outlet to collect the excess biofluid, and a plurality of plasmonic sensors; and
an encapsulation layer, wherein the encapsulation layer comprises an optically transparent material; and
quantifying the biofluid.

15. The method of claim 14, wherein the quantifying comprises simultaneous quantification of rate and volume of release and concentration of analytes in the biofluid, including pH, ions, metabolites, proteins, and pathogens.

16. The method of claim 14, wherein the quantifying comprises determining at least one of pH of the biofluid, rate of release from the subject of the biofluid, biofluid loss from the subject, volume of the biofluid, analytes in the biofluid, concentration of analytes in the biofluid, metabolites in the biofluid, concentration of metabolites in the biofluid, or combinations thereof.

17. The method of claim 14, wherein the quantifying comprises detection of at least one of analytes or metabolites in the biofluid.

18. The method of claim 14, wherein the biofluid comprises sweat, and wherein the quantifying comprises continuous quantitative analysis of sweat loss, sweat rate, and metabolites in sweat.

19. The method of claim 14, wherein the quantifying is performed via surface-enhanced Raman spectroscopy (SERS) or other optical approaches, colorimetric assay, enzyme-linked immunosorbent assay, fluorescence-linked immunosorbent assay, and combinations thereof.

20. The method of claim 14, wherein the plurality of plasmonic sensors comprise chromatography paper comprising a SERS substrate for stable SERS enhancement, wherein the SERS substrate comprises gold nanorods (AuNR).

21-22. (canceled)

23. The method of claim 14, wherein the encapsulation layer comprises at least one of polydimethylsiloxane (PDMS), a silicone adhesive, or a silicone adhesive layer sandwiched between PDMS encapsulation and the paperfluidic layer.
